(19) United States
(12) Patent Application Publication
(10) Pub. No.: US 2010/0125168 A1
(43) Pub. Date: May 20, 2010

(54) METHODS AND DEVICES FOR ENDOSCOPE CONTROL IN A BODY CAVITY

(75) Inventors: David Stefanchik, Morrow, OH (US); James T. Spivey, Cincinnati, OH (US); Omar J. Vakharla, Cincinnati, OH (US)

Correspondence Address: Ethicon Endo-Surgery/Nutter, McClennen & Fish LLP, World Trade Center West, 155 Seaport Blvd., Boston, MA, 02210-2604, US

(73) Assignee: Ethicon Endo-Surgery, Inc., Cincinnati, OH (US)

(21) Appl. No.: 12/271,072

(22) Filed: Nov. 14, 2008

(51) Int. Cl. A61B 1/01; A61B 1/00

(52) U.S. Cl. 600/114; 600/146

(57) ABSTRACT

Methods and devices are provided for controlling an endoscope in a body cavity. In one exemplary embodiment an endoscopic surgical system is provided that includes an endoscope and a steering tether coupled to at least a portion of the endoscope. The steering tether is configured to be manipulatable to effect directional movement of a distal end of the endoscope. The steering tether can include a loop formed on a distal end thereof such that a proximal portion of the steering tether can control the loop, which in turn controls the movement of the distal end of the endoscope. An overtube can optionally be included in the system. The overtube can be coupled to endoscope and adapted to receive at least a portion of the endoscope and the steering tether. In one embodiment, the endoscope includes an accessory mount extending over at least a portion of a length of the endoscope and the steering tether can be disposed on the accessory mount. Various methods for controlling movement of an endoscope in a body cavity are also provided.

### FIELD

The present disclosure relates to devices and methods for controlling an endoscope in a body cavity.

### BACKGROUND

Minimally invasive surgical techniques such as endoscopies and laparoscopies are often preferred over traditional open surgeries because the recovery time, pain, and surgery-related complications are typically less with minimally invasive surgical techniques. Rather than cut open large portions of the body in order to access inner cavities, such as the peritoneal cavity, surgeons either rely on natural orifices of the body or create one or more small orifices in which surgical instruments can be inserted to allow surgeons to visualize and operate at the surgical site. Surgeons can then perform a variety of diagnostic procedures, such as visual inspection or removal of a tissue sample for biopsy, or treatment procedures, such as removal of a polyp or tumor or restructuring tissue.

Because of the rise in popularity of minimally invasive surgeries, there has been significant development with respect to the instruments used in such procedures. These instruments need to be suitable for precise placement of a working end at a desired surgical site to allow the surgeon to see the site and perform the necessary actions at such site. Often times the instruments either themselves contain a device that allows the surgeon to see the site, or else the instruments are used in conjunction with an instrument that can provide visual assistance. At least one of these types of devices, an endoscope, is typically configured with both a lens to visualize the surgical site and one or more channels through which instruments can be delivered to the surgical site for subsequent use. The instruments themselves can be used to engage and or treat tissue and other portions within the body in a number of different ways to achieve a diagnostic or therapeutic effect.

Like most surgical procedures, minimally invasive procedures require stability and precision at the surgical site. This can be particularly difficult to achieve in body cavities because body cavities generally include a large amount of three-dimensional space, which in turn means that there is not much in the way of support within the cavity that the endoscope can rely upon for strength and stability. It is also challenging to remotely control a working end of an endoscope such that it can be directed to the desired location within the body cavity so that the desired procedures can be performed upon reaching the desired location. Further, the challenges of controlling an endoscope can also be experienced just trying to deliver the endoscope to the desired location. For example, organs or other materials in the body can get in the way of a desired path of the endoscope, in which case it is desirable to move the organs and materials out of the desired path without causing unwanted disruption to the organs and/or materials.

Accordingly, there remains a need for improved devices and methods for controlling endoscopes, and in particular the working end of endoscopes, to allow for more precision and accuracy during surgical procedures.

### SUMMARY

Methods and devices are generally provided for controlling an endoscope in a body cavity. In one embodiment, an endoscopic surgical system includes an endoscope, an overtube configured to receive at least a portion of the endoscope, and a steering tether disposed external to the endoscope, coupled to at least a portion of the endoscope, and configured to be manipulatable to effect directional movement of a distal end of the endoscope. The steering tether can extend through the overtube from a proximal end thereof and can be coupled to a portion of the endoscope that is proximal to the distal end of the endoscope. The endoscope can include an accessory mount extending over at least a portion of a length of the endoscope and terminating proximal to the distal end of the endoscope. The steering tether can be disposed on the accessory mount. In one embodiment a portion of the steering tether extends beyond the accessory mount to form a loop and another portion of the steering tether extends back through the overtube to a proximal end of the endoscope. The loop can include a nominal arcuate diameter that is adjustable such that when the nominal arcuate diameter is adjusted, a range of access of the steering tether, and thereby a range of access of the endoscope, can be altered. Similarly, in embodiments that do not include an accessory mount, a portion of the steering tether can extend beyond the distal end of the endoscope to form a loop and another portion of the steering tether can extend back through the overtube to a proximal end of the endoscope. The loop can include an adjustable nominal arcuate diameter as already described. Regardless of whether the steering tether includes a loop or not, it can be configured to be pushed and pulled to effect directional movement of the distal end of the endoscope. The steering tether can also be manipulatable to displace objects to a surrounding area above or below a plane of the endoscope. The endoscopic surgical system can include a locking mechanism to set a position of the steering tether. In one embodiment the endoscopic surgical system includes a steering module that is configured to adjust a position of the steering tether.

In another embodiment of an endoscopic surgical system, an endoscope and a steering tether are provided. The steering tether can be external to the endoscope and can be configured to couple to the endoscope. The steering tether can include a first portion that extends along side at least a portion of the endoscope and can be coupled to at least a portion of the endoscope, and a second portion that extends along side the first portion. A loop can be formed between the first and second portions. The loop can terminate proximal to a distal end of the endoscope and at least a portion of the loop can be coupled to the endoscope. The second portion of the steering tether can include a proximal portion that is configured to manipulate the loop to control movement of the endoscope. For example, the proximal portion can be configured to be pushed and pulled to control movement of the endoscope. The proximal portion can also be configured to be rotated about its longitudinal axis to displace objects to a surrounding area above or below a plane of the endoscope. The endoscopic surgical system can include a channel that has at least a portion of each of the endoscope and the steering tether disposed within it. In one embodiment the endoscope includes an accessory mount that extends over at least a portion of a length of the endoscope and terminates proximal to the distal end of the endoscope. At least part of the first portion of the steering tether can reside in the accessory mount. The loop can extend beyond the accessory mount. The loop can include a nominal arcuate diameter that is adjustable such that when the nominal arcuate diameter is adjusted, a range of access of the steering tether, and thereby a range of access of the endoscope, can be altered. In one embodiment the endoscopic surgical system includes a steering module that is configured to adjust a position of the steering tether.

In an embodiment of a surgical system, an elongate sheath having an accessory mount and a steering tether. The accessory mount of the elongate sheath can extend over at least a portion of a length of the accessory mount, and the accessory mount can terminate proximal to a distal end of the sheath. The steering tether can be disposed external to the elongate sheath, extend along at least a portion of the elongate sheath that is proximal to the distal end of the sheath, and can be manipulatable to effect directional movement of the distal end of the sheath. In one embodiment, at least a portion of the steering tether is disposed in the accessory mount. A portion of the steering tether can extend beyond the accessory mount to form a loop, while another portion of the steering tether can extend back through at least a portion of the accessory mount to a proximal end of the elongate sheath. An overtube can be coupled to the elongate sheath and at least a portion of each of the elongate sheath and the steering tether can be disposed therein. The system can also include an accessory channel coupled to the elongate sheath by way of a complimentary accessory mount that can be coupled to the accessory mount of the elongate sheath.

Methods for controlling movement of an endoscope in a body cavity are also provided. In one exemplary embodiment, an endoscope and a steering tether can be directed to a body cavity. Both the endoscope and the steering tether can be at least partially disposed within a channel and at least a portion of the steering tether can be coupled to the endoscope. The steering tether can include a proximal end and a looped distal end such that moving the proximal end of the steering tether can control the looped distal end, thereby controlling the directional movement of the endoscope. In one embodiment, moving the proximal end of the steering tether includes pushing and/or pulling it to move the looped distal end to a desired location. In another embodiment, moving the proximal end of the steering tether includes rotating it about a longitudinal axis of the steering tether to displace objects to a surrounding area above or below a plane of the endoscope. A nominal arcuate diameter of the looped distal end can be adjusted, and thus the method can include adjusting the nominal arcuate diameter of the looped distal end to adjust a range of access of the steering tether, and thereby the endoscope. In one embodiment, the method can also include directing a rail to a body cavity. The rail can be coupled to the endoscope, and thus, can be at least partially disposed in the channel. The rail can be configured to assist in moving the endoscope to a desired location.

### BRIEF DESCRIPTION OF DRAWINGS

This invention will be more fully understood from the following detailed description taken in conjunction with the accompanying drawings, in which:

FIG. 1 is a side perspective view of one exemplary embodiment of an endoscopic surgical system;

FIG. 2 is a further side perspective view of the endoscopic surgical system of FIG. 1;

FIG. 3 is a perspective view of the proximal end of the endoscopic surgical system of FIG. 1;

FIG. 4 is a perspective view of an exemplary embodiment of a surgical system that includes a sheath and an accessory channel;

FIG. 5 is a top perspective view of the endoscopic surgical system of FIG. 1 illustrated with respect to a polar coordinate grid;

FIG. 6 is a top perspective view of the endoscopic surgical system of FIG. 5 with an adjustable nominal arcuate diameter of a steering tether that is smaller than the nominal arcuate diameter of the system of FIG. 5;

FIG. 7 is a top perspective view of the endoscopic surgical system of FIG. 6 illustrating movement of a distal end of an endoscope of the system by way of the steering tether;

FIG. 8 is a distal end perspective view of the endoscopic surgical system of FIG. 1 having an object at least partially disposed in a plane of the endoscope of the system;

FIG. 9 is a partially transparent distal end perspective view of the endoscopic surgical system of FIG. 8 illustrating the system displacing the object from the plane of the endoscope of the system; and

FIGS. 10A-10F illustrate a progression of a method for controlling movement of an endoscope.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

An endoscopic surgical system is generally provided that includes an endoscope having a distal end configured to be accurately controlled from a proximal end of the surgical system. Unlike many endoscopes steering and/or directional movement can be controlled even in the absence of the walls of a hollow organ within which an endoscope is often placed. Directional movement generally describes any number of directions or orientations that the endoscope, and in particular the distal end of the endoscope, can be directed to move by an operator. The system can include a steering tether that is external to the endoscope, extends along at least a portion of the endoscope, and that can be manipulated to effect directional movement of the endoscope's distal end. While the steering tether can come in a variety of forms and have a number of different configurations as discussed herein, in one exemplary embodiment the tether forms a loop near the distal end of the endoscope such that the steering tether extends back to a proximal end of the endoscope and/or a proximal end of the surgical system. The system can optionally include an overtube that is configured to receive at least a portion of the endoscope and the steering tether. When the steering tether forms a loop, the overtube can receive two portions of the steering tether—the portion extending from the proximal end of the system toward the distal end of the endoscope to form the loop and the portion extending from the loop and returning toward the proximal portion of the system. A person having ordinary skill in the art would recognize that while many embodiments are discussed with respect to a portion of an endoscope being manipulated by a steering tether, other surgical instruments or tools, or other devices configured to receive surgical instruments or tools, such as a sheath, can be associated with a steering tether in a similar fashion as the endoscopes discussed herein, and further, the steering tether can be operated in a similar manner as described herein.

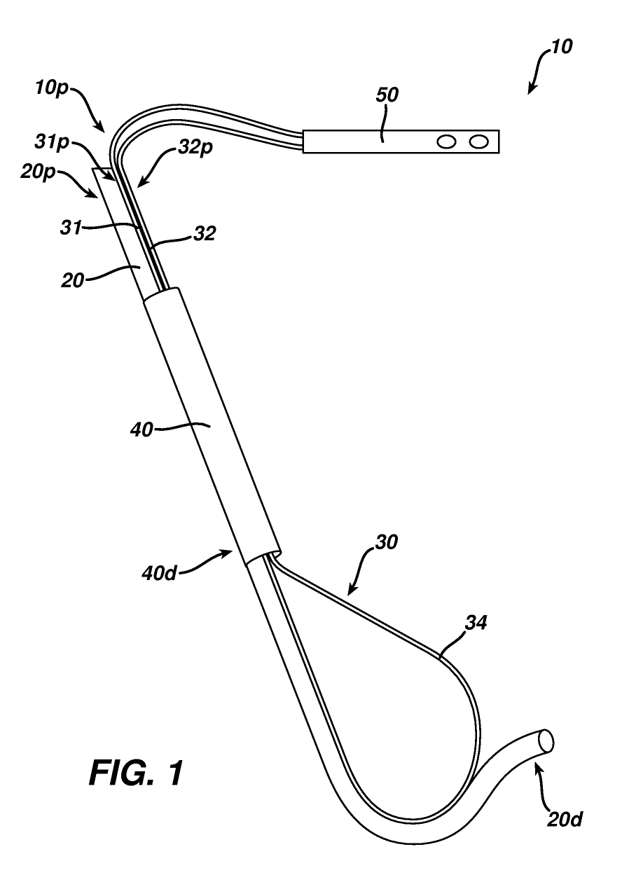

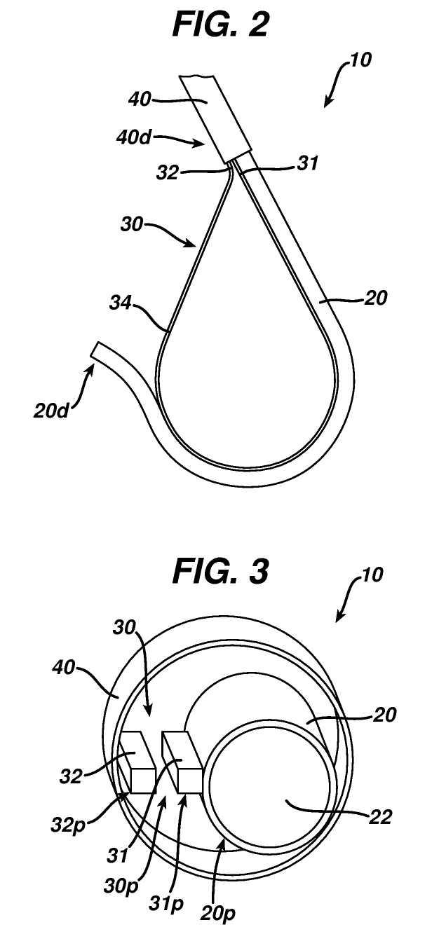

FIGS. 1-3 illustrate one exemplary embodiment of an endoscopic surgical system 10 configured to allow for accurate movement of a distal end of an endoscope. The system 10 can include a number of different components, but in the illustrated embodiment it includes an endoscope 20 and a steering tether 30 at least portions of which are disposed in an overtube 40. The overtube 40 can be configured to receive at least a portion of both the endoscope 20 and the steering tether 30. The steering tether 30 can be external to the endoscope 20, coupled to at least a portion of the endoscope 20, and further, it can be manipulatable to effect directional movement of a distal end 20d of the endoscope 20.

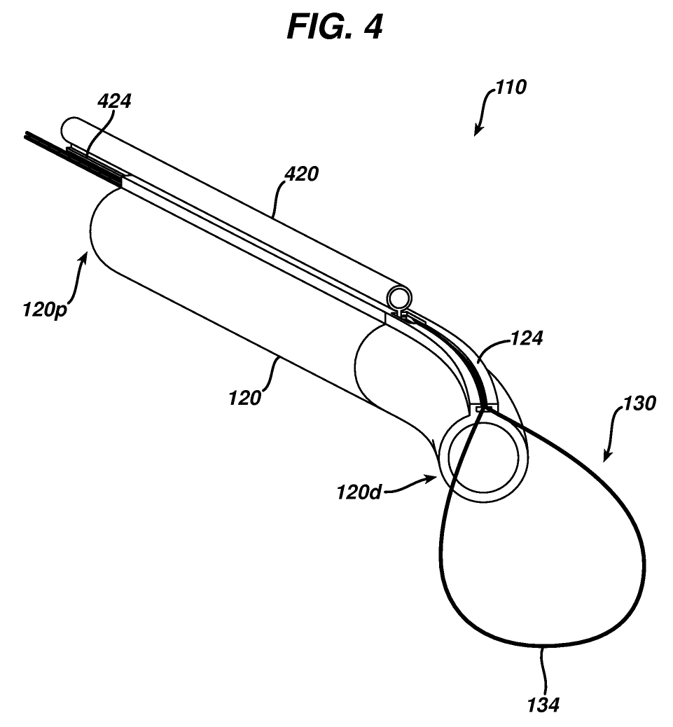

One skilled in the art will appreciate that endoscopes have many different configurations, and thus, an endoscope for use with the endoscopic surgical system 10 can have many different configurations. As shown in FIG. 3, the endoscope 20 can have a working channel 22 configured to receive one or more surgical instruments, or even the steering tether 30, at a proximal end 20p of the endoscope 20. In other embodiments, the endoscope 20 can include multiple working channels or have other configurations for use in surgical procedures. In still other embodiments of an endoscope that can be used in an endoscopic surgical system as discussed herein, the endoscope can include a mating element or accessory mount formed directly or indirectly on the endoscope and adapted to mate with another device, such as an elongate sheath or another endoscope. A mount formed indirectly on the endoscope may be, for example, a mount formed on a sheath within which an endoscope can be disposed.

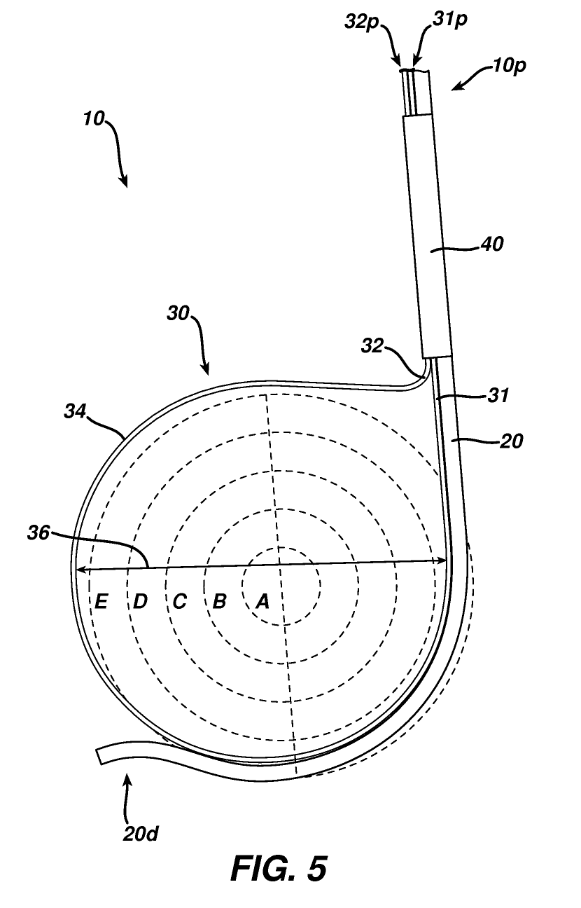

While the mating element or accessory mount can have a variety of configurations, including for example interlocking elements, engaging elements, complementary shapes, sliding members, magnetic elements, spring-loaded retaining members, and elastic members, in the embodiment of a surgical system 110 illustrated in FIG. 4 the mating element is a T-shaped track 124 formed along at least a portion of an external length of an elongate sheath 121, which can house an endoscope (not shown). In some embodiments, the sheath is an endoscope. The sheath 121 can have characteristics and features that are similar to the endoscope 20 of the endoscopic surgical system 10. In the illustrated embodiment, the track 124 extends across the entire length of the sheath 121, terminating at a distal end 121d of the sheath 121, although in other embodiments the track 124 can terminate proximal to the distal end 121d of the sheath 121 or be positioned along any portion thereof. The track 124 is configured to be slidably mated to a complimentary rail 424 of a second device, such as accessory channel 420. The track 124 can have virtually any length, but in one exemplary embodiment the length compliments the length of the sheath 121 so that a second device can be securely mated thereto. Allowing a second device or channel to be disposed next to the sheath 121 can allow other tools to be delivered adjacent to the distal end 121d of the sheath 121. By way of non-limiting example, the independent articulating accessory channel discussed in U.S. Patent Application Publication No. 2008/0132758 of Stefanchik et al., filed on Dec. 5, 2006, and entitled “Independent Articulating Accessory Channel,” which is incorporated by reference in its entirety, is one type of configuration in which the teachings of a steering tether like steering tethers 30 and 130, both of which are discussed in greater detail below, can be incorporated. Likewise, mechanisms for controlling any portion of the sheath 121 can then be disposed along any length of the sheath 121. For example, devices that assist in stiffening the sheath 121, or instruments disposed therein, either entirely or portions thereof, can be disposed along a length of the sheath 121 by way of the mating element. By way of non-limiting example, stiffening elements such as those discussed in U.S. patent application Ser. No. 11/952,475 of Stefanchik et al., filed on Dec. 7, 2007, and entitled “Selective Stiffening Devices and Methods,” which is hereby incorporated by reference in its entirety, can be used in association with the surgical systems disclosed herein. By further way of non-limiting example, other embodiments of endoscopes that can be used in the surgical systems disclosed herein are discussed in U.S. Patent Application Publication No. 2008/0183035 of Vakharia et al., filed on Jan. 26, 2007, and entitled “Endoscopic Accessory Control Mechanism,” and in U.S. patent application Ser. No. 11/971,410 of Stefanchik et al., filed on Jan. 9, 2008, and entitled “Articulating Surgical Device and Method of Use,” each of which is hereby incorporated by reference in its entirety. While in the illustrated embodiment the track 124 is disposed along an external length of the sheath 121 and is configured to be slidably mated to the complimentary rail 424 of the accessory channel 420, in alternative embodiments the sheath 121 can include a rail while a complimentary track, like the track 124, can be disposed on a second device. Still further, mating elements or accessory mounts of the sheath 121 can be configured to mate with portions of a steering tether 130 or an overtube (not shown).

Referring again to FIGS. 1-3, the steering tether 30 is an elongate member that is configured to control the distal end 20d of the endoscope 20, for example by being external to the endoscope 20 and/or being coupled to at least a portion of the endoscope 20. In one embodiment, the steering tether is coupled to a portion of the endoscope 20 that is proximal to the distal end 20d of the endoscope 20. As shown, the steering tether 30 is coupled to the proximal end 20p of the endoscope 20 and remains coupled to the endoscope 20 along the length of the endoscope 20 up to a portion that is proximal of the distal end 20d of the endoscope 20. The steering tether 30 can thus dovetail with a length of the endoscope 20. In alternative embodiments, select portions of the steering tether 30 are coupled to select portions of the endoscope 20, which can allow for varying degrees of control of the endoscope 20. Still further, although in the illustrated embodiment the steering tether 30 is external to the endoscope 20, in other embodiments portions of the steering tether 30 can be disposed in the endoscope 20 such that a portion or segment of the steering tether 30 is internal to the endoscope 20 while a second portion or segment of the steering tether 30 is external to the endoscope 20.

One skilled in the art will appreciate that different configurations of associating the steering tether 30 with the endoscope 20 can allow a variety of directional movement of the distal end 20d of the endoscope 20 to be effected. As the steering tether 30 is manipulated, by techniques discussed in further detail below including, for example, pushing and pulling the tether 30, the distal end 20d of the endoscope 20 is controlled. In the illustrated embodiment, the steering tether 30 is configured such that a loop 34 is formed at a distal end 30d thereof. The loop 34 can be manipulated at a proximal end 30p of the steering tether 30. In one embodiment, the loop 34 can be formed between a first portion 31 of the steering tether 30 that is coupled to at least a portion of the endoscope 20 and a second portion 32 of the steering tether 30 that can extend along side the first portion 31 and can include a proximal portion 32p that is configured to manipulate the loop 34. Manipulation of the loop 34 can control directional movement of the endoscope 20. The proximal portion 32p can be controlled in a variety of ways, such as by manipulating a portion of the proximal end 30p of the steering tether 30, or alternatively, the system 10 can include a steering module 50 (FIG. 1). The steering module 50 can be adapted to control movement of the proximal portion 32p of the steering tether 30, thereby controlling directional movement of the distal end 20d of the endoscope 20. In one embodiment the steering module 50 is operated by an operator during the course of a procedure. In another embodiment the steering module 50 is programmed to operate autonomously, e.g., programmed to make one or more desired movements. Ideally the system 10 is operable from a proximal end 10p thereof.

While in the described embodiment the proximal portion 32p of the second portion 32p of the steering tether 30 is configured to manipulate the loop 34, in other embodiments a proximal portion 31p of the first portion 31 of the steering tether 30 can be configured to manipulate the loop 34 or both of the proximal portions 31p, 32p can be configured to manipulate the loop 34 independently, cooperatively, or simultaneously. The proximal portion 31p can be controlled in the same ways in which the proximal portion 32p can be controlled, including, by way of non-limiting example, via the steering module 50.

While one exemplary configuration of the steering tether 30 includes a loop, in other configurations a loop need not be used. One skilled in the art will appreciate other configurations that would also be effective to cause directional movement in the distal end 20d of the endoscope 20 by manipulating the steering tether 30. By way of non-limiting example, the steering tether 30 cab be a string coupled at or near the distal end 20d of the endoscope 20. The string can be pushed and pulled to control the movement of the distal end 20d of the endoscope 20. In one embodiment, the string is tensioned. Still other configurations could also be used, which are contemplated and able to be adapted to effect directional movement of the distal end 20d of the endoscope 20 from the proximal end 10p of the system 10.

It is important to note that while the steering tether 30 is configured to control the distal end 20d of the endoscope 20, it can perform its functions in lieu of, in conjunction with, and/or in addition to traditional mechanisms and means used to control endoscope movement. The steering tether 30 provides a mechanical advantage outside of the endoscope to enable desired control of the distal end 20d of the endoscope 20, while traditional mechanisms, such as steering wires disposed within the endoscope 20, can provide some means of control as well. As discussed herein, however, traditional mechanisms are constrained by the fact that they generally do not operate well in large cavities in which the endoscope is unable to rely on walls of lumens to control movement thereof.

As illustrated, the endoscopic surgical system 10 can optionally include a channel or overtube 40 that is configured to receive at least a portion of the endoscope 20 and/or one or more portions of the steering tether 30. In one embodiment each of the first and second portions 31, 32 of the steering tether 30 extend through the overtube 40. As shown, the loop 34 is formed distal of the overtube 40, directly after the steering tether 30 exits a distal end 40d of the overtube 40, although the loop 34 can be formed from any portion of the steering tether 30 and in any location with respect to the overtube 40, such as, for example, within the overtube 40 or at a location distal of a location directly past the distal end 40d of the overtube 40. The overtube 40 can be coupled to the endoscope 20 to provide a rigid and stable location through which the steering tether 30 can pass. It can be desirable to slidably mate the overtube 40 with the endoscope 20 such that the rigid and stable location through which the steering tether 30 can pass can be adjusted as desired. Further, changing the location of the overtube 40 can be effective to change a nominal arcuate diameter of the loop 34, the effect of which will be discussed in further detail below. In some embodiments, only one portion 31, 32 of the steering tether 30 is disposed in the overtube 40, and still in other embodiments the overtube 40 does not house the steering tether 30 at all, either because it has other instruments disposed therein or because it is not included in the system 10. Generally the overtube 40 is rigid and stiff, and it can be made of a variety of materials, such as polymers. In one exemplary embodiment the overtube 40 is made of Teflon Polyethylene Nylon.

As shown in FIG. 4, in embodiments that include an accessory mount, such as the track 124, the steering tether 130 can be disposed in the accessory mount. Alternatively, the steering tether 130 can be disposed on the accessory mount. The steering tether 130 can have characteristics and features that are similar to the steering tether 30 of the endoscopic surgical system 10. Accordingly, similar to coupling the steering tether 30 with the endoscope 20, the steering tether 130 can be coupled to any portion of the track 124, including the entire portion, or alternatively, select portions of the steering tether 130 can be coupled to select portions of the track 124. In the illustrated embodiment a portion of the steering tether 130 extends beyond the track 124 in forming a loop 134, while another portion of the steering tether 130 extends through the track 124 and back toward a proximal end 121p of the sheath 121. Although not illustrated, in other embodiments, an overtube, similar to the overtube 40 as described above, can be mounted to the sheath 121. Similar to the overtube 40 of the endoscope 20, the overtube in embodiments that include an accessory mount can optionally be part of the surgical system 110 and can have characteristics and features that are similar to the overtube 40 of the surgical system 10. The overtube can be located along any portion of the sheath 121, including at a location proximal to the distal end 121d approximately where the loop 134 terminates, it can be slidable to assist in changing the capabilities and range of access of the steering tether 130, and it can allow at least a portion of a second device, like the accessory channel 420, to be disposed therein. While an overtube can be mounted to the sheath 121, it can also be mounted to the accessory mount, such as track 124. A person having ordinary skill in the art could apply the teachings related to the overtube 40 to an embodiment including an accessory mount, like the surgical system 110, without difficulty.

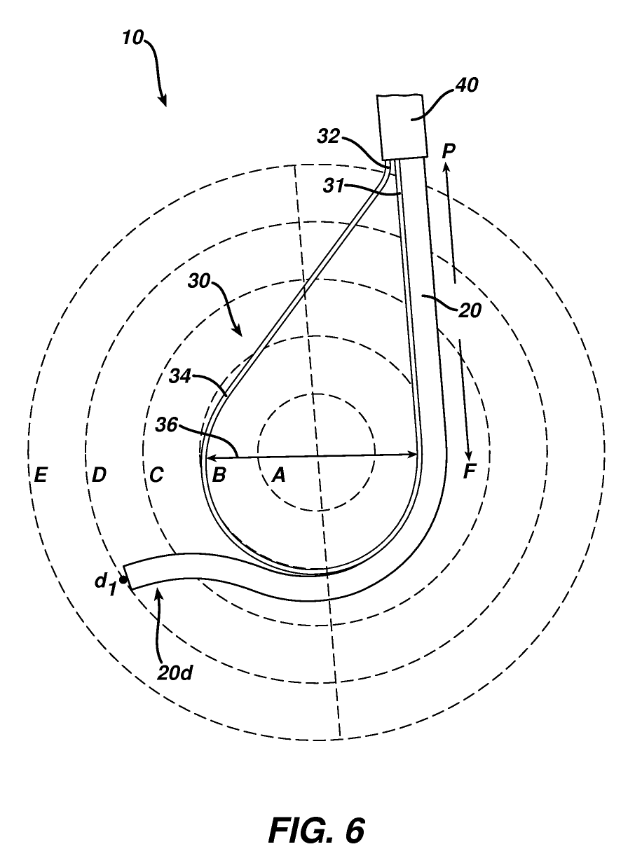

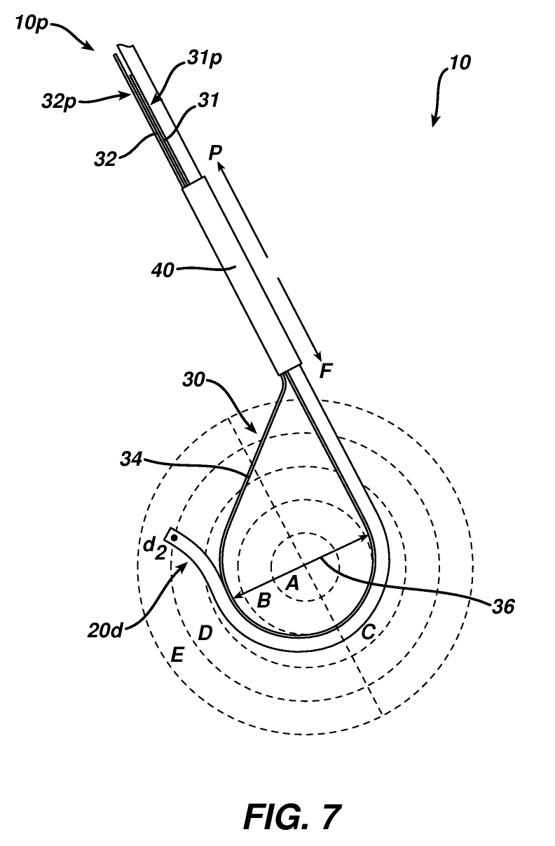

In embodiments in which the steering tether of an endoscopic surgical system is a loop, a size of the loop can be adjusted to affect directional movement and/or a range of access of the endoscope. A range of access generally describes a finite number of locations that can be reached via directional movements. As a range of access is adjusted, a new finite number of locations can be achieved via directional movements. The finite number of locations between various ranges of access can overlap. With specific references to the loop 34, as the loop 34 is made bigger and smaller, the locations within the body that the distal end 20d of the endoscope 20 can reach changes, as does the directional movements that can be made by the distal end 20d of the endoscope 20. As illustrated in FIGS. 5 and 6, the loop 34 of the steering tether 30 includes a nominal arcuate diameter 36 that can be adjusted. One skilled in the art will appreciate that although the loop 34 is discussed with respect to having a nominal arcuate diameter, to the extent that it has any non-circular shape, an equivalent to the nominal arcuate diameter can easily be determined. One skilled in the art will also appreciate that the nominal arcuate diameter 36 can be adjusted in a variety of ways, such as, by way of non-limiting example, adjusting a location of the overtube 40, but in one exemplary embodiment at least one of the first and second portions 31, 32 can be moved with respect to each other to adjust the nominal arcuate diameter 36. Similar to manipulating the loop 34, proximal portions 31p, 32p of the first and second portions 31, 32 can be configured to operate independently, cooperatively, or simultaneously to adjust the nominal arcuate diameter 36 of the loop 34. Adjusting the nominal arcuate diameter 36 adjusts a range of access of the steering tether 30, which in turn adjust a range of access of the endoscope 20 because the tether 30 is manipulatable to effect directional movement of the distal end 20d of the endoscope 20.

FIG. 5 illustrates the system 10 with respect to a polar grid in which the nominal arcuate diameter 36 is relatively large and is approximately circular in shape. More particularly, the nominal arcuate diameter 36 is approximately the same size as the diameter of the largest circle E of the polar grid, although as illustrated a portion of the endoscope 20 proximal to the distal end 20d sits inside the circle E and a portion of the loop 34 that is opposite of this portion of the endoscope 20 sits outside of the circle E. FIG. 6 also illustrates the system 10 with respect to the polar grid, but the nominal arcuate diameter 36 of the steering tether 30 is smaller than in FIG. 5 and is approximately in the shape of a tear-drop. More particularly, the nominal arcuate diameter 36 is approximately the same size as the diameter of circle B of the polar grid. Of course, the nominal arcuate diameter 36 can be any number of sizes, and in fact the loop 34 can take on a number of different shapes as the nominal arcuate diameter 36 is adjusted. For example, the nominal arcuate diameter 36 can be adjusted to cover a range of access between 180 and 360 degrees. Further, the nominal arcuate diameter 36 can be adjusted prior to disposing the steering tether 30 at a surgical site, while the steering tether 30 is being delivered to the surgical site, or after it has been delivered to the surgical site.

Once a nominal arcuate diameter 36 of a desired size is achieved, it can be locked in place using a locking mechanism (not shown). One skilled in the art will appreciate that many different locking mechanisms can be suitable for locking the adjustable nominal arcuate diameter 36, such as by way of non-limiting example a knob that can move between unlocked and locked positions. The choice of locking mechanism can be based at least in part on how the nominal arcuate diameter 36 is configured to adjust. In one embodiment the locking mechanism holds the first and second portions 31, 32 approximately stationary with respect to each other. Further, the locking mechanism can be located at any portion of the tether 30, for example at the proximal end 10p of the system 10, at a portion distal of the overtube 40, or as part of the steering module 50.

A locking mechanism can also be used to hold the location of the tether 30 and/or the endoscope 20 once it reaches a desired location, methods of which are discussed in further detail below. The locking mechanism for holding a location of one or more components of the system 10 such as the tether 30 and the endoscope 20 can be similar to the locking mechanism for holding the nominal arcuate diameter 36 of the loop 34 and such teachings can be easily adapted for use with a locking mechanism for holding the location of components of the system 10. In one embodiment, both the locking mechanism for holding the nominal arcuate diameter 36 of the loop 34 and the locking mechanism for holding the location of the tether 30 and/or the endoscope 20 can be one in the same. Each of the two described locking mechanisms can be adapted to cooperate with each other.

The steering tether 30 can be made from a variety of materials and have a variety of sizes. Preferably the steering tether 30 is semi-rigid, semi-flexible, or flexible. Many polymers can be used to provide the desired flexibility. In one embodiment of a semi-flexible tether 30, polyethylene is used to form the tether 30. In another embodiment of a semi-flexible tether 30, polytetrafluoroethylene, e.g., Teflon, is used to form the tether 30. The tether 30 can likewise have any length and thickness, but in one embodiment the length can be approximately in the range of 100 to 300 cm, and more particularly can be approximately 200 cm, while a thickness in one embodiment can be approximately in the range of about 1 to 10 mm, and more particularly can be approximately 6 mm.

In use, the endoscopic surgical system 10 is designed so that the distal end 20d of the endoscope 20 can be moved to a number of different locations by manipulating the steering tether 30. This is at least partially because while the endoscope 20 is generally good in torsion, it does not generally curl, and thus the steering tether 30 can use the existing torque stiffness to help maneuver the endoscope 20. The steering tether 30 can provide a mechanical advantage or leverage to assist in controlling the endoscope 20. The steering tether can be manipulated in a number of different ways, which are based at least in part on the configuration of the steering tether and its association with the endoscope. In one embodiment the proximal portion 32p of the second portion 32 of the steering tether 30 can be manipulated to effect the desired directional movement of the distal end 20d of the endoscope 20. In other embodiments, the proximal portion 31p of the first portion 31 of the steering tether 30 can be manipulated to effect the desired directional movement of the distal end 20d of the endoscope. The proximal portions 31p, 32p can be operated individually, cooperatively, and/or simultaneously as desired.

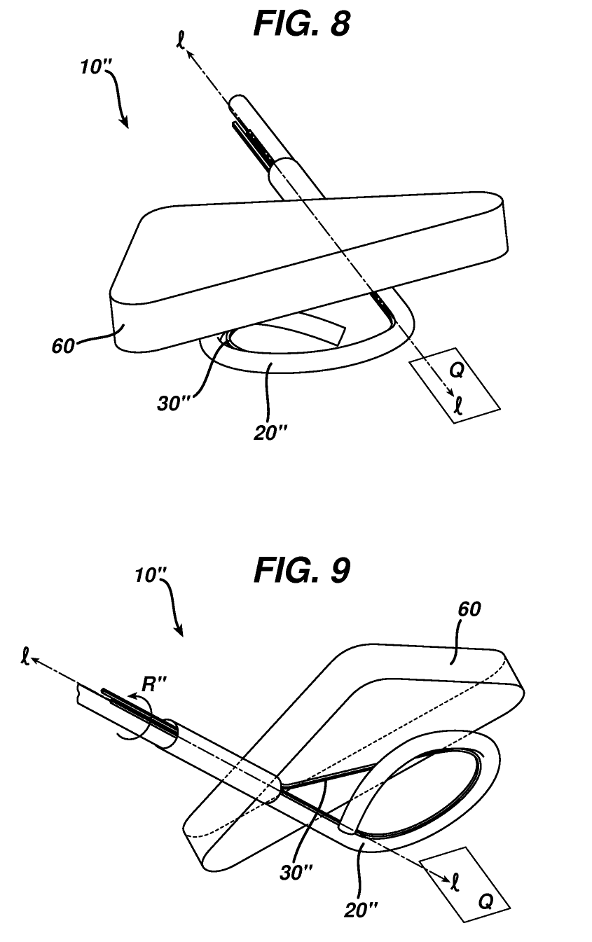

As illustrated by FIGS. 6 and 7, two ways of manipulating the steering tether 30 are by pushing and pulling it. As shown, pulling on the proximal portion 32p of the steering tether 30 in a direction P can move the distal end 20d of the endoscope 20 from a first position, illustrated by FIG. 6, in which the distal end 20d is located at a position d₁ on a circle D of the polar grid, toward the proximal portion 10p of the system 10 to a second position, illustrated by FIG. 7, in which the distal end 20d is located at a position d₂ on the circle D of the polar grid. Likewise, pushing on the proximal portion 32p of the steering tether 30 in a direction F can move the distal end 20d of the endoscope 20 from the second position to the first position. One skilled in the art will appreciate that the illustrated positions are just examples of locations to which the steering tether 30 can move the distal end 20d, and that any number of positions can be achieved by manipulating the steering tether 30, as discussed in more detail above. Further, the positions that the distal end 20d can reach can be affected, at least in part, by adjusting a range of access of the steering tether 30, as also discussed in more detail above, such as, for example, by adjusting the nominal arcuate diameter 36 of the loop 34 of the steering tether 30.

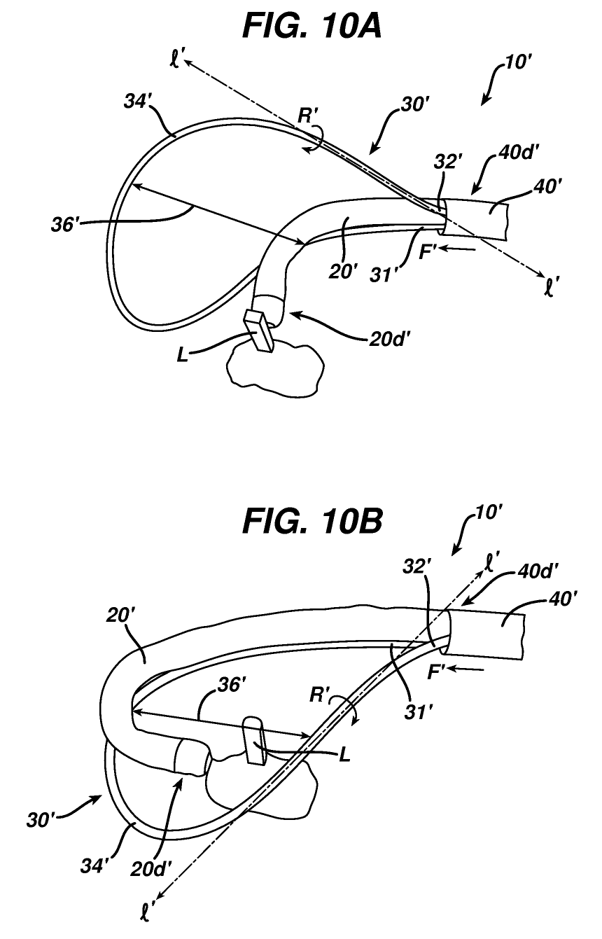

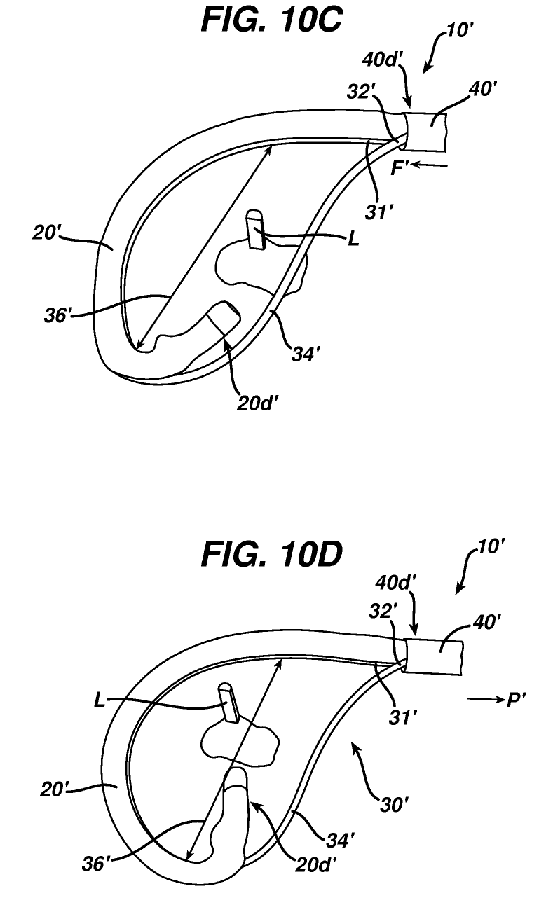

The steering tether can also be manipulated to allow the endoscopic surgical system to displace objects to a surrounding area above or below a plane of an endoscope. As illustrated in FIGS. 8 and 9, the steering tether can be twisted or rotated about its longitudinal axis 1, which in turn can lift objects out of a desired pathway. In FIG. 8, an object 60, representative of an organ or other component of a body, is disposed in a desired pathway of an endoscopic surgical system 10″, which is similar to the endoscopic surgical system 10. More particularly, at least a portion of the object 60 is disposed in a plane Q. As illustrated in FIG. 8, the plane Q is substantially aligned with a surface of an endoscope 20″, and the desired pathway extends through and past the portion of the object 60 that is disposed in the plane Q. In order to move the object 60 from the desired pathway, the endoscopic surgical system 10″ can be brought into the vicinity of the object 60 such that manipulation can place the endoscope 20″ in contact with the object 60. In the illustrated embodiment the endoscope 20″ is moved directly into contact with the object 60, but in other embodiments, the endoscope 20″ can be manipulated, such as by twisting, and/or rotating a steering tether 30″, to move the endoscope 20″ into contact with the object 60 once the endoscope 20″ is in the vicinity of the object 60. As shown in FIG. 9, once the endoscope 20″ is in contact with the object 60, it can be rotated in a direction R″ out of the plane Q, thereby displacing the object 60 from the plane Q. After displacing the object 60, the endoscope 20″ and the steering tether 30″ can be returned approximately to the plane Q and/or the desired pathway as appropriate, or a second device, such as a second endoscopic surgical system, can be directed to the desired pathway as desired by the operator.

Still further, as discussed with respect to FIGS. 5 and 6 above, the nominal arcuate diameter 36 of the loop 34 can be adjusted as desired. Adjusting the nominal arcuate diameter 36 can be done on its own or it can be done in conjunction and/or simultaneously with some of the other manipulations of the steering tether 30, 30″ discussed above. More particularly, it can be desirable to use any combination of pushing, pulling, twisting, and rotating of the steering tether 30, and adjusting the nominal arcuate diameter 36 of the loop 34 of the steering tether 30, in any sequence and in any combination, including performing more than one of the manipulations at the same time.

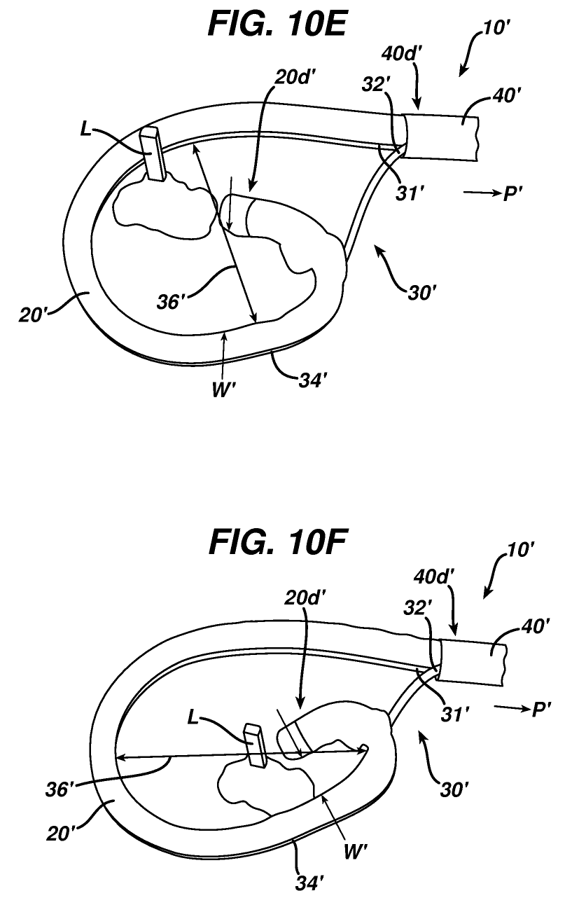

FIGS. 10A-10F illustrate one example of a progression of an endoscopic surgical system 10′ in use. The components of the surgical system 10′ are similar to the components discussed with respect to the endoscopic surgical systems 10 and 10″. The progression shows how a steering tether 30′ can be used to guide a distal end 20d′ of an endoscope 20′ around a desired location L. As shown in FIG. 10A, the endoscope 20′ and the steering tether 30′ are at least partially disposed in an overtube 40′. A first portion 31′ of the steering tether 30′ is coupled to the endoscope 20′ along a length thereof and a second portion 32′ of the steering tether 32′ extends along side the first portion 31′ within the overtube 40′. A loop 34′ is formed between the first and second portions 31′, 32′. The loop 34′ extends beyond the endoscope 20′ and terminates proximal to the distal end 20d′ of the endoscope 20′, more particularly approximately at a distal end 40d′ of the overtube 40′. Pushing a proximal portion (not illustrated) of the second portion 32′ of the steering tether 30′ in a direction F′ and rotating the same in a direction R′ about its longitudinal axis l′ allows the distal end 20d′ of the endoscope 20′ to be moved to the location illustrated in FIG. 10B. As shown, the distal end 20d′ is now distal of the location L and the loop 34′ encircles the location L to allow the distal end 20d′ to begin to wrap around the desired location L. The steering tether 30′ can be pushed further in the direction F′, as shown in FIG. 10C, to allow more of the endoscope 20′ to be distal of the desired location L, thereby allowing more of the endoscope 20′ to eventually be wrapped around the location L. As shown in FIG. 10D, the proximal portion (not illustrated) of the second portion 32′ of the steering tether 30′ can be pulled in the direction P′ to begin moving the distal end 20d′ of the endoscope 20′ toward the desired location L. In the illustrated embodiment the distal end 20d′ is no longer distal of the desired location L, but rather, is approximately in line with the desired location L. This action also allows the endoscope 20′ as a whole to encircle the desired location L more fully. Further pulling in the direction P′ can move the distal end 20d′ proximal of the desired location L, as shown in FIG. 10E. In the embodiment illustrated in FIG. 10E, a portion of the endoscope 20′ that is proximal of the distal end 20d′ is pulled closer to the desired location L and a nominal arcuate diameter 36′ of the loop 34′ is decreased, thus allowing the endoscope 20′ to almost fully encircle the desired location L. As shown in FIG. 10F, the distal end 20d′ can be bent further by pulling the proximal portion (not illustrated) of the second portion 32′ in the direction P′ and adjusting the nominal arcuate diameter 36′ of the loop 34′. More particularly, a distance W′ between the distal end 20d′ of the endoscope 20′ and a portion proximal of the distal end 20d′ of the endoscope 20′ in FIG. 10E is greater than the distance W′ in FIG. 10F.

One skilled in the art will appreciate that the progression described with respect to FIGS. 10A-10F is only one of a myriad of ways in which methods can be performed that use the devices described herein. Any combination of manipulation steps can be used to control movement of an endoscope in a body cavity. Various amounts of pushing, pulling, twisting, and rotating any portion of a steering tether, and in embodiments in which the steering tether includes a loop, adjusting a nominal arcuate diameter of the loop, can be used to effect the desired directional movement of a distal end of an endoscope. Further, making minor changes to the design of the endoscopic surgical system can also cause the methods performed to be adjusted, and one skilled in the art, relying on the disclosures herein, would be able to apply various manipulation techniques to such endoscopic surgical systems.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An endoscopic surgical system, comprising:
an endoscope;
an overtube configured to receive at least a portion of the endoscope; and
a steering tether extending through the overtube from a proximal end thereof and being disposed external to the endoscope and coupled to at least a portion of the endoscope proximal to a distal end of the endoscope, the steering tether being manipulatable to effect directional movement of the distal end of the endoscope.

2. The system of claim 1, wherein the endoscope includes an accessory mount extending over at least a portion of a length thereof, terminating proximal to the distal end of the endoscope, and wherein the steering tether is disposed on the accessory mount.

3. The system of claim 2, wherein a portion of the steering tether extends beyond the accessory mount to form a loop and another portion of the steering tether extends back through the overtube to a proximal end of the endoscope.

4. The system of claim 3, wherein the loop further comprises an adjustable nominal arcuate diameter, wherein adjusting the adjustable nominal arcuate diameter of the loop affects a range of access of the steering tether and thereby a range of access of the endoscope.

5. The system of claim 1, wherein a portion of the steering tether extends beyond the distal end of the endoscope to form a loop and another portion of the steering tether extends back through the overtube to a proximal end of the endoscope.

6. The system of claim 5, wherein the loop further comprises an adjustable nominal arcuate diameter, wherein adjusting the nominal arcuate diameter of the loop affects a range of access of the steering tether and thereby a range of access of the endoscope.

7. The system of claim 1, wherein the steering tether is manipulatable to displace objects to a surrounding area above or below a plane of the endoscope.

8. The system of claim 1, further comprising a steering module configured to adjust a position of the steering tether.

9. An endoscopic surgical system comprising:
an endoscope; and
a steering tether disposed external to the endoscope and configured to couple thereto, comprising:
a first portion extending along side at least a portion of the endoscope and being coupled to at least a portion thereof;
a second portion extending along side the first portion and having a proximal portion; and
a loop formed between the first and second portions, the loop terminating proximal to a distal end of the endoscope and at least a portion of the loop being coupled to the endoscope;
wherein the proximal portion of the second portion is configured to manipulate the loop to control movement of the endoscope.

10. The system of claim 9, further comprising a channel having at least a portion of each of the endoscope and steering tether disposed therein.

11. The system of claim 10, wherein the endoscope includes an accessory mount extending over at least a portion of the length thereof, terminating proximal to the distal end of the endoscope, and wherein at least part of the first portion of the steering tether resides in the accessory mount.

12. The system of claim 11, wherein the loop of the steering tether extends beyond the accessory mount.

13. The system of claim 9, wherein the proximal portion of the second portion of the steering tether is configured to be pushed and pulled to control movement of the endoscope.

14. The system of claim 9, wherein the proximal portion of the second portion of the steering tether is configured to be rotated about its longitudinal axis to displace objects to a surrounding area above or below a plane of the endoscope.

15. The system of claim 9, wherein the loop further comprises an adjustable nominal arcuate diameter, wherein adjusting the nominal arcuate diameter of the loop affects a range of access of the steering tether and thereby a range of access of the endoscope.

16. A surgical system comprising:
an elongate sheath having an accessory mount extending over at least a portion of a length thereof, terminating proximal to a distal end of the sheath;
a steering tether disposed external to the elongate sheath and extending along at least a portion of the elongate sheath that is proximal to the distal end of the sheath, the steering tether being manipulatable to effect directional movement of the distal end of the sheath.

17. The surgical system of claim 16, wherein at least a portion of the steering tether is disposed in the accessory mount.

18. The surgical system of claim 17, wherein a portion of the steering tether extends beyond the accessory mount to form a loop and another portion of the steering tether extends back through at least a portion of the accessory mount to a proximal end of the elongate sheath.

19. The surgical system of claim 18, further comprising an overtube coupled to the elongate sheath, the overtube having at least a portion of each of the elongate sheath and the steering tether disposed therein.

20. The surgical system of claim 16, further comprising an accessory channel coupled to the elongate sheath by way of a complimentary accessory mount coupled to the accessory mount of the elongate sheath.
